(19) United States
(12) Patent Application Publication
(10) Pub. No.: US 2022/0185077 A1
(43) Pub. Date: Jun. 16, 2022

(54) VEHICLE AIR CONDITIONER EQUIPPED WITH PHOTOCATALYST

(71) Applicants: HYUNDAI MOTOR COMPANY, Seoul (KR); KIA CORPORATION, Seoul (KR); Hanon Systems, Daejeon (KR)

(72) Inventors: Dong Ho KWON, Yongin-si (KR); Gee Young SHIN, Suwon-si (KR); Myung Hoe KIM, Seoul (KR); Seung Sik HAN, Hwaseong-si (KR); Jae Ho KIM, Daejeon (KR); Ji Yong PARK, Daejeon (KR)

(73) Assignees: HYUNDAI MOTOR COMPANY, Seoul (KR); KIA CORPORATION, Seoul (KR); Hanon Systems, Daejeon (KR)

(21) Appl. No.: 17/342,778

(22) Filed: Jun. 9, 2021

(30) Foreign Application Priority Data
Dec. 14, 2020 (KR) 10-2020-0173966

(51) Int. Cl. B60H 3/06

(52) U.S. Cl. CPC B60H 3/0633; B60H 2003/0675

(57) ABSTRACT

A vehicle air conditioner is equipped with a photocatalyst which is irradiated with light so that bacteria generated in the photocatalyst is removed. In particular, an entire area of the photocatalyst is irradiated with the light using a small number of light sources so that sterilization effect of a filter can be improved.

### CROSS REFERENCE TO RELATED APPLICATION

This application claims priority to and the benefit of Korean Patent Application No. 10-2020-0173966, filed on Dec. 14, 2020, the entire contents of which are incorporated herein by reference.

### FIELD

The present disclosure relates to a vehicle air conditioner equipped with a photocatalyst, which sterilizes a filter for filtering foreign materials in air-conditioned air.

### BACKGROUND

The statements in this section merely provide background information related to the present disclosure and may not constitute prior art.

An air conditioning device is provided in a vehicle to provide air-conditioned air to an interior of the vehicle. A duct through which the air-conditioned air circulates in the air conditioning device is provided with a filter for filtering foreign materials included in the air-conditioned air, of which a temperature is controlled, to provide clean air.

The air-conditioned air continuously passes through the filter. When air-conditioned air including moisture passes through the filter, condensation is formed in the filter. In this case, various microorganisms and bacteria in the air-conditioned air multiply in the condensation to generate a stench. As described above, when the filter is contaminated, air-conditioned air including fungi or bacteria is provided to the interior of the vehicle to causes harm to a body of an occupant and generate a stench so that inconvenience may occur to the occupant.

The foregoing is intended merely to aid in understanding of the background of the present disclosure, and is not intended to mean that the present disclosure falls within the purview of the related art that is already known to those skilled in the art.

### SUMMARY

The present disclosure proposes a vehicle air conditioner equipped with a photocatalyst, which removes bacteria from a filter for filtering foreign materials in air-conditioned air to provide clean air to an interior of a vehicle.

According to one aspect of the present disclosure, a vehicle air conditioner includes: a housing in which air flows in and out by a blower; a photocatalyst module fixed to a path through which air flows in the housing and equipped with a photocatalyst which responds to light energy to remove a harmful substance; and a light source installed on an inner side of the photocatalyst module to emit light to the photocatalyst, provided as a plurality of light sources to be disposed to be spaced a predetermined interval apart from each other, disposed such that light irradiation areas partially overlap or are in contact with each other, and configured to allow an entirety of the photocatalyst to be irradiated with the light.

A plurality of frames which extend to cross inward the photocatalyst module and in which the plurality of light sources are installed may be formed to be spaced apart from each other in the photocatalyst module, and the plurality of frames and the photocatalyst may be disposed to be spaced apart from each other in an air flow direction.

The plurality of light sources may be disposed to allow the light irradiation areas to partially overlap and to be disposed to not pass over a center of adjacent light irradiation area.

Each of the plurality of light sources may be installed further upstream in the air flow direction than the photocatalyst.

A first installation frame and a second installation frame, which extend to cross inward the photocatalyst module, may be formed on an upper side and a lower side in the photocatalyst module, respectively, based on the photocatalyst, and a plurality of first light sources and a plurality of second light sources may be installed in the first installation frame and the second installation frame, respectively, so that an upper portion and a lower portion of the photocatalyst may be irradiated with the light.

The plurality of first light sources may be disposed to be spaced apart from each other to allow light irradiation areas thereof to be in contact with each other, the plurality of second light sources may be disposed to be spaced apart from each other to allow light irradiation areas thereof to be in contact with each other, and a light irradiation area of one among the plurality of second light sources may be disposed to overlap light irradiation areas of two or more among the plurality of first light sources.

The first installation frame and the second installation frame may be disposed to be laterally spaced apart from each other so as to not vertically coincide with each other.

The photocatalyst may be formed of double or multiple layers.

The photocatalyst may include a first photocatalyst and a second photocatalyst which are disposed to be vertically spaced apart from each other, and the light source may include a plurality of first light sources for emitting light to the second photocatalyst and a plurality of second light sources for emitting light to the first photocatalyst.

A first connection frame which extends to cross the first photocatalyst and in which the first light source is installed, and a second connection frame which extends to cross the second photocatalyst and in which the second light source installed may be formed in the photocatalyst module.

The first connection frame may be provided as a plurality of first connection frames to be disposed to be laterally spaced apart from each other so that the first photocatalyst may be provided between the first connection frames, and the second connection frame may be provided as a plurality of second connection frames to be disposed to be laterally spaced apart from each other so as to not vertically coincide with the first connection frames so that the second photocatalyst may be provided between the second connection frames.

The plurality of first light sources may be disposed to be spaced apart from each other to allow a light irradiation area to be formed in the second photocatalyst between the plurality of second connection frames, and the plurality of second light sources may be disposed to be spaced apart from each other to allow a light irradiation area to be formed in the first photocatalyst between the plurality of first connection frames.

A filter configured to filter foreign materials in air may be provided in the housing, and the photocatalyst module and the light source may be installed further downstream in the air flow direction than the filter.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a diagram illustrating a vehicle air conditioner equipped with a photocatalyst according to one form of the present disclosure;

FIGS. 2 and 3 are diagrams respectively illustrating a photocatalyst module and a photocatalyst of the vehicle air conditioner equipped with a photocatalyst shown in FIG. 1;

FIGS. 4 and 5 are diagrams for describing a first form of the present disclosure;

FIGS. 6 and 7 are diagrams for describing a second form of the present disclosure; and

FIG. 8 is a diagram for describing a third form of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

Hereinafter, a vehicle air conditioner equipped with a photocatalyst according to exemplary forms of the present disclosure will be described with reference to the accompanying drawings.

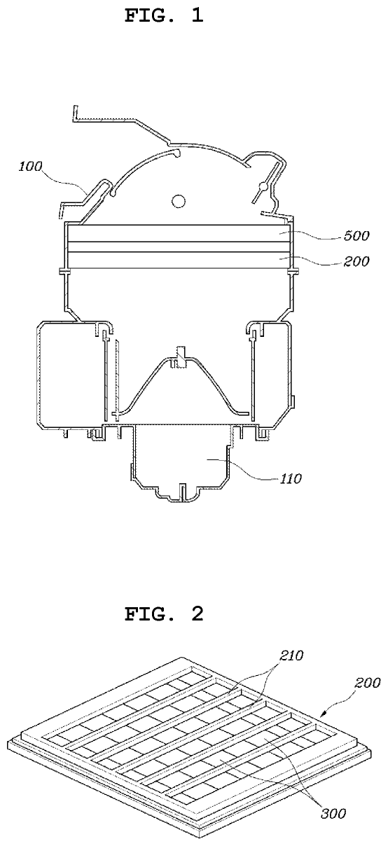

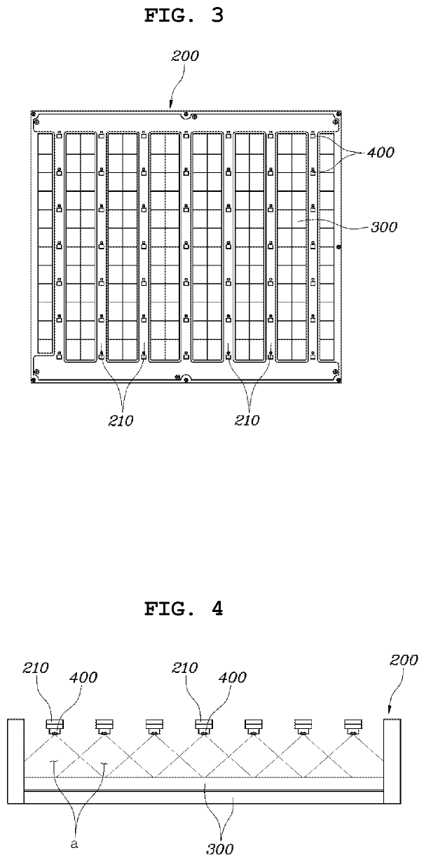

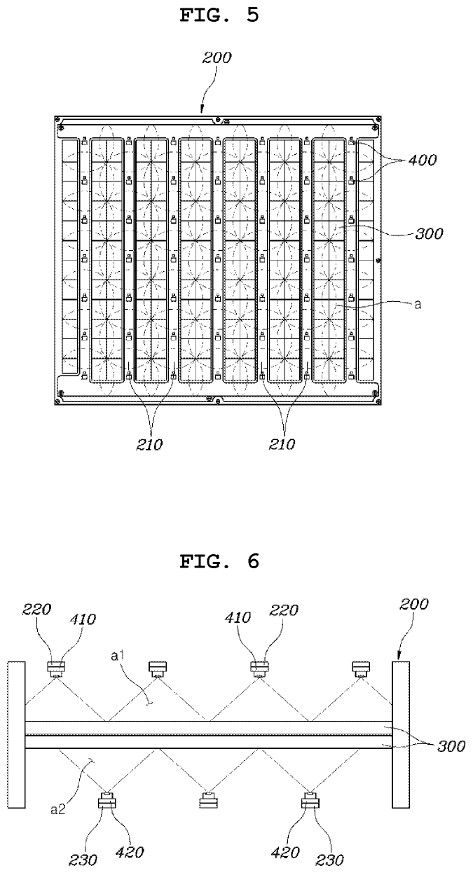

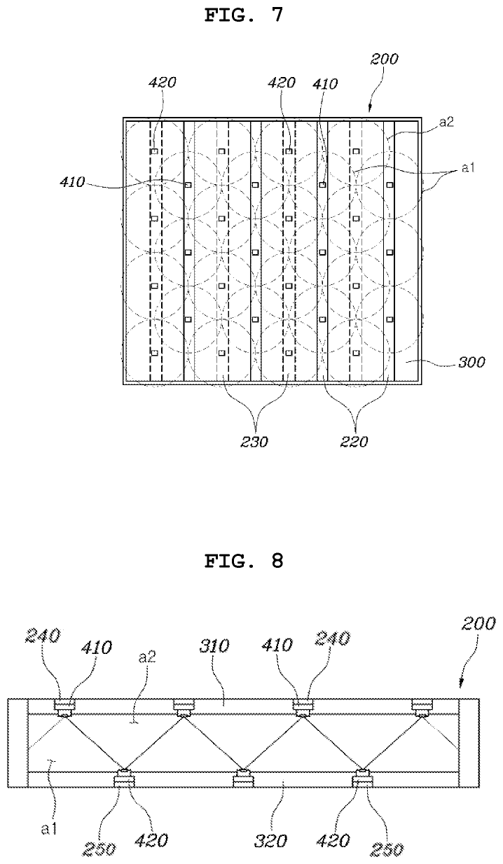

FIG. 1 is a diagram illustrating a vehicle air conditioner equipped with a photocatalyst according to one form of the present disclosure, FIGS. 2 and 3 are diagrams illustrating a photocatalyst module and a photocatalyst of the vehicle air conditioner equipped with a photocatalyst shown in FIG. 1, FIGS. 4 and 5 are diagrams for describing a first form of the present disclosure, FIGS. 6 and 7 are diagrams for describing a second form of the present disclosure, and FIG. 8 is a diagram for describing a third foam of the present disclosure.

As shown in FIGS. 1 to 3, the vehicle air conditioner equipped with a photocatalyst includes a housing 100 in which air flows in and out by a blower 110, a photocatalyst module 200 fixed to a path through which air flows in the housing 100 and equipped with a photocatalyst 300 which responds to light energy to remove a harmful substance; and a light source 400 installed on an inner side of the photocatalyst module 200 to emit light to the photocatalyst 300, provided as a plurality of light sources to be disposed to be spaced a predetermined interval apart from each other, disposed such that light irradiation areas a partially overlap or are in contact with each other, and configured to allow an entirety of the photocatalyst 300 to be irradiated with the light.

The photocatalyst 300 and the light source 400 are installed in the housing 100, in which the air flows in and out, through the photocatalyst module 200. In addition, a filter 500 for filtering foreign materials in air may be provided in the housing 100, and the photocatalyst module 200 and the light source 400 may be installed further downstream than the filter 500 in an air flow direction. Thus, when the blower 110 is operated in the housing 100 and thus the air passes through the filter 500, foreign materials are removed from the air, and then the air passes through the photocatalyst 300. Here, the photocatalyst 300 is fixed in the housing 100 through the photocatalyst module 200 and formed to fill an interior of the housing 100, and thus the air flowing in the housing 100 passes through the photocatalyst 300. A plurality of fine cells are formed in the photocatalyst 300 to allow air to pass therethrough and are coated with a photocatalyst material. Here, the photocatalyst material may be composed of TiO₂ which is made to remove bacteria without being varied even when exposed to light.

Meanwhile, the light source 400 for emitting light to the photocatalyst 300 is provided as a plurality of light sources 400 in the photocatalyst module 200, and the plurality of light sources 400 are disposed to be spaced a predetermined interval from each other to emit light to an entirety of the photocatalyst 300. In particular, the light sources 400 are disposed such that the light irradiation areas a partially overlap or are in contact with each other. Thus, the entirety of the photocatalyst 300 is irradiated with the light without an area in which the light from the light source 400 is not incident on the photocatalyst 300. Consequently, sterilization due to irradiation of the light is performed on an entire area of the photocatalyst 300 so that the sterilization effect is improved.

The above described present disclosure may be applied to various forms as follows.

As shown in FIG. 4, as a first form, a plurality of frames 210 which are formed to extend and cross inward and in which the plurality of light sources 400 are installed are formed to be spaced apart from each other in the photocatalyst module 200, and the plurality of frames 210 and the photocatalyst 300 are disposed to be spaced apart from each other in the air flow direction.

As described above, the plurality of frames 210 are spaced apart from each other to extend and cross inward the photocatalyst module 200 so that overall rigidity of the photocatalyst module 200 is reinforced. In particular, since the light sources 400 are installed through the plurality of frames 210 disposed in the photocatalyst module 200, each of the light sources 400 may emit light toward the photocatalyst 300. In addition, since the frames 210 and the photocatalyst 300 are disposed to be spaced apart from each other in the air flow direction, the light emitted from the light source 400 may be projected onto the photocatalyst 300 with the light irradiation area a.

In particular, the plurality of light sources 400 may be disposed to allow the light irradiation areas “a” to partially overlap and disposed to not pass over a center of each of the light irradiation areas “a”. As described above, the plurality of light sources 400 are disposed to allow the light irradiation areas “a” to partially overlap so that the entire area of the photocatalyst 300 is irradiated with the light without a separation gap. However, the light irradiation area a of each of the light sources 400 is disposed to not pass over a center of a light irradiation area “a” of another adjacent light source 400 so that the number of the disposed light sources 400 is reduced and efficiency of light energy of the light sources 400 is improved. That is, the photocatalyst 300 is sterilized when light energy is incident thereon. However, the sterilization effect is not continuously increased in proportion to an incident amount of the light energy, but a certain degree of the sterilization effect is maintained. Thus, the light sources 400 are disposed to allow the light irradiation areas “a” to partially overlap and to not pass over the centers of the adjacent light irradiation areas “a” so that unnecessary waste of light energy due to multiple overlapping of the light irradiation areas “a” of the light sources 400 is inhibited or prevented. In addition, since a separation distance between of the light sources 400 is secured, the number of the light sources 400 may also be reduced, and light energy may be efficiently secured.

Meanwhile, the light sources 400 may be installed further upstream in the air flow direction than the photocatalyst 300. Assuming that the air moves from an upper side to a lower side of the housing 100, foreign materials and bacteria are collected on an upper portion of the photocatalyst 300. Thus, since the light sources 400 are installed upstream in the air flow direction than the photocatalyst 300, and thus the upper portion of the photocatalyst 300 is irradiated with the light, contaminants may be efficiently removed during the sterilization of the photocatalyst 300.

As shown in FIG. 5, in the above described first form, since the light irradiation areas “a” of the light sources 400 overlap and are projected onto the entire area of the photocatalyst 300, the sterilization may be performed on the entire area of the photocatalyst 300. In addition, the photocatalyst 300 is formed to overlap in double or multiple layers so that filtering performance and sterilization performance for foreign materials may be improved.

As shown in FIG. 6, as a second form, a first installation frame 220 and a second installation frame 230, which extend to cross inward a photocatalyst module 200, are formed in an upper side and a lower side of the photocatalyst 300, respectively, and a plurality of first light sources 410 and a plurality of second light sources 420 are installed in the first installation frame 220 and the second installation frame 230, and thus the upper portion and the lower portion of the photocatalyst 300 are irradiated with light.

The first installation frame 220 and the second installation frame 230 may be provided as a plurality of first installation frames 220 and a plurality of second installation frames 230 to be disposed on an inner side of the photocatalyst module 200 and to be spaced apart from each other on the upper side and the lower side of the photocatalyst 300 to extend to cross inward the photocatalyst module 200. Thus, overall rigidity of the photocatalyst module 200 is reinforced. In particular, the first light sources 410 are installed in the first installation frames 220 and thus the upper portion of the photocatalyst 300 is irradiated with light, and the second light sources 420 are installed in the second installation frames 230 and thus the lower portion of the photocatalyst 300 is irradiated with light. Thus, the photocatalyst 300 may receive light energy and thus sterilization may be performed. As described above, the light energy is incident on the upper and lower portions of the photocatalyst 300, thereby increasing self-sterilization performance of the photocatalyst 300.

Here, the first light sources 410 are disposed to be spaced apart from each other to allow light irradiation areas “a1” thereof to be in contact with each other, and the second light sources 420 are disposed to be spaced apart from each other to allow light irradiation areas “a2” thereof to be in contact with each other. The light irradiation area “a2” of one second light source 420 may be disposed to overlap the light irradiation areas “a1” of two or more first light sources 410. Thus, the light irradiation area “a1” of the one first light source 410 may be disposed to overlap the light irradiation areas “a2” of the two or more second light sources 420.

As described above, the light irradiation areas “a1” of the first light sources 410 are in contact with each other, and the light irradiation areas “a2” of the second light sources 420 are in contact with each other so that the entire area of the photocatalyst 300 is irradiated with the light without a separation gap. In addition, since the light irradiation area “a1” of the first light source 410 is disposed to partially overlap the light irradiation area “a2” of the second light source 420, a separation gap between the light irradiation areas “a1” of the first light sources 410 or between the light irradiation areas “a2” of the second light sources 420 is filled so that sterilization is performed on the entire area of the photocatalyst 300 to secure sterilization performance.

Here, the first installation frame 220 and the second installation frame 230 may be disposed to be laterally spaced apart from each other so as to not vertically coincide with each other. That is, as shown in FIG. 7, since the first installation frame 220 and the second installation frame 230 are disposed to be laterally spaced apart from each other, the first light source 410 and the second light source 420 are also disposed to be laterally spaced apart from each other. With this arrangement, in the photocatalyst 300, a separation gap between the light irradiation area “a1” of the first light source 410 and the light irradiation area “a2” of the second light source 420 does not occur in an area in which air flows. In addition, since the light irradiation area “a1” of the first light source 410 partially overlaps the light irradiation area “a2” of the second light source 420 in a portion in which the air flows in the photocatalyst 300, the sterilization performance of the photocatalyst 300 is secured.

Thus, the photocatalyst 300 may be formed to overlap in double or multiple layers. That is, in the case of the second form, since the first light sources 410 and the second light sources 420 transmit light energy to the upper portion and the lower portion of the photocatalyst 300, respectively, the photocatalyst 300 is formed of double or multiple layers to allow the sterilization performance to be increased. In addition, since the photocatalyst 300 is provided as a plurality of photocatalysts 300, filtering performance thereof with respect to foreign materials is also improved.

As described above, as shown in FIG. 7, in the second form, the light irradiation area “a1” of the first light source 410 overlaps the light irradiation area “a2” of the second light source 420 and thus the light is projected onto the upper portion and the lower portion of the photocatalyst 300, which are portions in which air flows. Thus, sterilization may be smoothly performed on the photocatalyst 300.

Meanwhile, as shown in FIG. 8, as a third form, a photocatalyst 300 may include a first photocatalyst 310 and a second photocatalyst 320 which are disposed to be vertically spaced apart from each other, and a light source 400 may include a plurality of first light sources 410 for emitting light to the second photocatalyst 320 and a plurality of second light sources 420 for emitting light to the first photocatalyst 310.

As described above, the first photocatalyst 310 and the second photocatalyst 320 are formed in the photocatalyst module 200, thereby securing filtering performance with respect to foreign materials and sterilization performance due to light energy. In addition, since the first photocatalyst 310 and the second photocatalyst 320 receive light energy from the first light source 410 and the second light source 420, respectively, it is easy to form a light irradiation area “a2” on the first photocatalyst 310 and a light irradiation area “a1” on the second photocatalyst 320.

Specifically, a first connection frame 240 which extends to cross the first photocatalyst 310 and in which the first light source 410 is installed, and a second connection frame 250 which extends to cross the second photocatalyst 320 in which the second light source 420 is installed are formed in a photocatalyst module 200.

The first connection frame 240 and the second connection frame 250 may be provided as a plurality of first connection frames 240 and a plurality of second connection frames 250 to be disposed on an inner side of the photocatalyst module 200 so that overall rigidity of the photocatalyst module 200 is reinforced. In addition, each of the first connection frames 240 extends to cross the first photocatalyst 310 so that the first photocatalyst 310 may be coupled between the first connection frames 240, and each of the second connection frames 250 extends to cross the second photocatalyst 320 so that the second photocatalyst 320 may be coupled between the second connection frames 250. In addition, since the first light source 410 is installed in the first connection frame 240 and the second light source 420 is installed in the second connection frame 250, an entire package of the photocatalyst module 200 is reduced.

Here, the first connection frame 240 is provided as a plurality of first connection frames 240 to be disposed to be laterally spaced apart from each other so that the first photocatalyst 310 may be provided between the first connection frames 240, and the second connection frame 250 is provided as a plurality of second connection frames 250 to be disposed to be laterally spaced apart from each other so as to not vertically coincide with the first connection frame 240 so that the second photocatalyst 320 may be provided between the second connection frames 250.

As described above, since the first connection frame 240 and the second connection frame 250 are disposed to be laterally spaced apart from each other, the first light source 410 and the second light source 420 are also disposed to be laterally spaced apart from each other. With this arrangement, in the photocatalyst 300, a separation gap between the light irradiation area “a1” of the first light source 410 and the light irradiation area “a2” of the second light source 420 does not occur in an area in which air flows.

Here, the first light sources 410 are disposed to be spaced apart from each other to allow a light irradiation area “a1” to be formed in the second photocatalyst 320 between the second connection frames 250, and the second light sources 420 are disposed to be spaced apart from each other to allow a light irradiation area “a2” to be formed in the first photocatalyst 310 between the first connection frames 240. Thus, the first light sources 410 may be disposed to allow light irradiation areas “a1” to be in contact with each other, and the second light sources 420 may be disposed to allow light irradiation areas “a2” to be in contact with each other. In addition, the first light source 410 and the second light source 420 may not be disposed to be collinear with each other in a lateral direction and may be disposed in a zigzag manner.

As described above, the first photocatalyst 310 provided between the plurality of first connection frames 240 receives light energy from the second light source 420 installed in the second connection frame 250 and thus sterilization is performed on the first photocatalyst 310, and the second photocatalyst 320 provided between the plurality of second connection frames 250 receives light energy from the first light source 410 installed in the first connection frame 240 and thus sterilization is performed on the second photocatalyst 320. In addition, the first light source 410 and the second light source 420 correspond to the second photocatalyst 320 and the first photocatalyst 310, respectively, to emit light thereto, thereby securing sterilization performance with respect to the first photocatalyst 310 and the second photocatalyst 320.

The vehicle air conditioner equipped with a photocatalyst, which is formed in the above described structure, emits light to the photocatalyst 300, thereby removing bacteria generated in the photocatalyst 300. In addition, the entire area of the photocatalyst 300 is irradiated with the light using a small number of light sources 400 so that sterilization effect of the filter is improved.

In accordance with a vehicle air conditioner equipped with a photocatalyst, which is formed in the above described structure, a photocatalyst is irradiated with light so that bacteria generated in the photocatalyst can be removed. In addition, in accordance with the vehicle air conditioner equipped with a photocatalyst, an entire area of the photocatalyst is irradiated with the light using a small number of light sources so that sterilization effect of a filter can be improved.

Although exemplary forms of the present disclosure have been described for illustrative purposes, those skilled in the art will appreciate that various modifications, additions and substitutions are possible, without departing from the scope and spirit of the present disclosure.

What is claimed is:

1. A vehicle air conditioner, comprising:
a housing through which air flows in and out by a blower;
a photocatalyst module provided in a flow path through which the air flows into the housing, and including a photocatalyst which is activated with light energy to remove a harmful substance; and
a plurality of light sources installed on an inner side of the photocatalyst module and each configured to emit light to the photocatalyst,
wherein light sources of the plurality of light sources are disposed to be spaced apart from each other at a predetermined interval, such that light irradiation areas of the light sources partially overlap or are in contact with each other, and the light sources are configured to allow an entirety of the photocatalyst to be irradiated with the light.

2. The vehicle air conditioner of claim 1, wherein:
a plurality of frames configured to extend to cross the photocatalyst module,
the light sources are installed on the plurality of frames and spaced apart from each other; and
the plurality of frames and the photocatalyst are disposed to be spaced apart from each other in an air flow direction.

3. The vehicle air conditioner of claim 1, wherein the light sources are disposed to allow the light irradiation areas to partially overlap and disposed to not pass over a center of adjacent light irradiation area.

4. The vehicle air conditioner of claim 1, wherein each of the light sources is installed further upstream in an air flow direction than the photocatalyst.

5. The vehicle air conditioner of claim 1, wherein:
a filter configured to filter foreign materials in the air is provided in the housing; and
the photocatalyst module and the light sources are installed further downstream in an air flow direction than the filter.

6. A vehicle air conditioner, comprising:
a housing through which air flows in and out by a blower;
a photocatalyst module provided in a flow path through which the air flows into the housing, and including a photocatalyst which is activated with light energy to remove a harmful substance;
a first installation frame and a second installation frame, which extend to cross the photocatalyst module and are formed on an upper side and a lower side of the photocatalyst in the photocatalyst module, respectively; and
a plurality of first light sources and a plurality of second light sources, which are installed in the first installation frame and the second installation frame, respectively, such that an upper portion and a lower portion of the photocatalyst is irradiated with light emitted from the plurality of first light sources and second light sources.

7. The vehicle air conditioner of claim 6, wherein:
the plurality of first light sources are disposed to be spaced apart from each other and configured to allow light irradiation areas thereof to be in contact with each other;
the plurality of second light sources are disposed to be spaced apart from each other and configured to allow light irradiation areas thereof to be in contact with each other; and
a light irradiation area of one second light source among the plurality of second light sources is disposed to overlap light irradiation areas of two or more first light sources among the plurality of first light sources.

8. The vehicle air conditioner of claim 6, wherein the first installation frame and the second installation frame are disposed to be laterally spaced apart from each other so as to not vertically coincide with each other.

9. The vehicle air conditioner of claim 6, wherein the photocatalyst is formed of double or multiple layers.

10. A vehicle air conditioner, comprising:
a housing through which air flows in and out by a blower;
a photocatalyst module provided in a flow path through which the air flows into the housing, and including at least one photocatalyst which is activated with light energy to remove a harmful substance, wherein the at least one photocatalyst includes a first photocatalyst and a second photocatalyst disposed to be vertically spaced apart from each other; and
a plurality of first light sources configured to emit light to the second photocatalyst; and
a plurality of second light sources configured to emit light to the first photocatalyst,
wherein light irradiation areas of the plurality of first and second light sources partially overlap or are in contact with each other, and cover an entirety of the first and second photocatalysts.

11. The vehicle air conditioner of claim 10, further comprising:
at least one first connection frame configured to extend to cross the first photocatalyst, the plurality of first light sources installed on the at least one first connection frame; and
at least one second connection frame configured to extend to cross the second photocatalyst, and the plurality of second light sources installed on the at least one second connection frame in the photocatalyst module.

12. The vehicle air conditioner of claim 11, wherein:
the at least one first connection frame includes a plurality of first connection frames to be disposed to be laterally spaced apart from each other so that the first photocatalyst is provided between the plurality of first connection frames; and
the at least one second connection frame includes a plurality of second connection frames to be disposed to be laterally spaced apart from each other so as to not vertically coincide with the first connection frames and the second photocatalyst is provided between the plurality of second connection frames.

13. The vehicle air conditioner of claim 12, wherein:
first light sources of the plurality of first light sources are disposed to be spaced apart from each other to allow a light irradiation area to be formed in the second photocatalyst between the plurality of second connection frames; and
second light sources of the plurality of second light sources are disposed to be spaced apart from each other to allow a light irradiation area to be formed in the first photocatalyst between the plurality of first connection frames.
